(19) United States
(12) Patent Application Publication
(10) Pub. No.: US 2007/0197482 A1
(43) Pub. Date: Aug. 23, 2007

(54) Regulation of guanine nucleotide exchange factor for a protein belonging to the rap family of small gtpases

(75) Inventors: Ian McPhee, Glasgow (GB); Catherine Breslin, Glasgow (GB); Justin Peter Kewney, Glasgow (GB); Simon John MacKenzie, Glasgow (GB); Ann Cooreman, Glasgow (GB); Lucien Charles Donald Gibson, Glasgow (GB); Morag McFarlane, Glasgow (GB); Stephen Hammond, Glasgow (GB)

Correspondence Address: EDWARDS ANGELL PALMER & DODGE LLP, P.O. BOX 55874, BOSTON, MA, 02205, US

(73) Assignee: Scottish Biomedical Limited, Glasgow (GB)

(21) Appl. No.: 10/555,371

(22) PCT Filed: May 4, 2004
(86) PCT No.: PCT/GB04/01907

§ 371 Date: Jan. 5, 2007

(30) Foreign Application Priority Data
May 2, 2003 (GB) 0310174.8

(51) Int. Cl. A61K 31/655; A61K 31/5377; A61K 31/519; A61K 31/428; A61K 31/425; A61K 31/426; A61K 31/12; C07D 487/02; C07D 417/02

(52) U.S. Cl. 514/150; 514/369; 514/367; 514/235.2; 514/680; 514/259.41; 514/256; 548/152; 548/183; 544/281

(57) ABSTRACT

The invention relates generally to the methods of treating brain diseases and compounds for treating brain diseases, and more specifically relates to using compounds that are able to modulate guanine nucleotide exchange factors for proteins belonging to the Rap family of small GTPases to treat diseases of the brain, such as Alzheimer's and Schizophrenia.

The invention relates generally to the methods of treating brain diseases and compounds for treating brain diseases and, more specifically, relates to using compounds that are able to modulate guanine nucleotide exchange factors for proteins belonging to the Rap family of small GTPases, such as EPAC 1 and EPAC 2 to treat diseases of the brain, such as Alzheimer's. The invention may also be involved in the treatment of a number of other disease states.

Neurodegenerative diseases and neurological disorders cover a wide range of disease states, including a number of pathological states involving neuronal degeneration, such as Parkinson's Disease, HuntingtonHuntington's Disease and Alzheimer's Disease, as well as Amyotrophic Lateral Sclerosis (ALS). Other mental illnesses include Schizophrenia and general dementia. Alzheimer's Disease is one of the most commonly found neurodegenerative disorders in the elderly. The instance of these disease states continues to increase, possibly in relation to the increasing life spans of people, which creates serious public health issues. At the present time these disorders and other related neurological disorders are neither curable nor preventable.

Many neurological disorders show some heredity, signifying a genetic component to the diseases (although this seems to be less so in the case of Parkinson's), however, in many cases the diseases arise spontaneously in the population.

Focusing specifically on Alzheimer's, the disease is a progressive neurodegenerative disorder of the central nervous system. The symptoms of Alzheimer's are mainly attenuation and decline in memory. Alzheimer's Disease was originally defined as a pre-senile dementia, but it now appears that the same pathology underlines the dementia irrespective of the age onset. Therefore, the term dementia of the Alzheimer's type signifies all dementias that do not have an obvious organic cause, such as stroke, brain damage or alcohol. The prevalence of Alzheimer's and related dementias rises sharply from the age of about 60 years, reaching somewhere in the region of 90% by the age of 95. Dementias of the Alzheimer's type are associated with the general shrinkage of brain tissue, but with relatively little loss of cortical neurones. Two characteristics of the disease are the presence of amyloid plaques and the presence of neurofibrillary tangles. Although these characteristics appear in normal brains, this tends to be in smaller numbers.

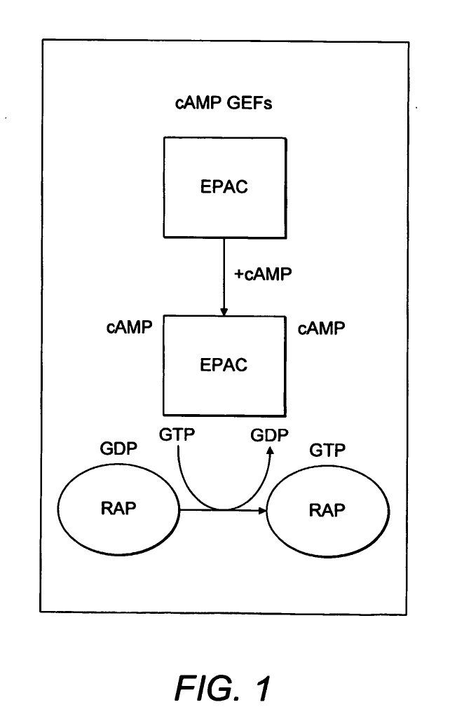

Work carried out by the inventors of the present invention has shown that EPAC 1 and EPAC 2 genes are strongly up-regulated and down-regulated respectively in Alzheimer's Disease. EPACs (Exchange Proteins directly Activated by cAMP) have only been discovered fairly recently (Kawasaki et al '98, A family of cAMP-binding proteins that directly activate Rap1, Science Vol 282(5397) p 2275-2279: de Rooji et al '98, Epac is a Rap1 guanine-nucleotide-exchange factor directly activated by cAMP. Nature Vol 396 p 474-477), and are known to be CAMP affected proteins which are widely expressed and have implications in a huge variety of cellular functions. Their discovery identified a new way for cAMP to exert effects upon the cell. FIG. 1 summarises the action of EPACs. At the present time, the exact nature of any involvement that the genes have in cellular functions has only recently begun to be investigated. So far, there has been no link between EPAC genes and Alzheimer's.

It can be seen that it would be beneficial to provide compounds to treat neurological or neurodegenerative disorders, such as Alzheimer's.

It can be seen that it would be beneficial to provide a screen to find compounds that are useful in the treatment of neurological or neurodegenerative disorders, such as Alzheimer's.

It is a first object of the present invention to provide a medicament for the treatment of neurological or neurodegenerative disorders, such as Alzheimer's, which comprises compounds that are able to regulate guanine nucleotide exchange factors, such as EPAC 1 and EPAC 2.

It is a further object of the present invention to provide a method of treatment for neurological and neurodegenerative disorders, such as Alzheimer's.

A yet further object of the present invention is to provide a method or methods of screening for compounds that are able to modulate or regulate guanine nucleotide exchange factors, such as EPAC 1 and EPAC 2, which therefore may be useful as therapeutic compounds for the treatment of neurological and neurodegenerative disorders, such as Alzheimer's.

Throughout this document reference to EPAC relates to both EPAC 1 and EPAC 2.

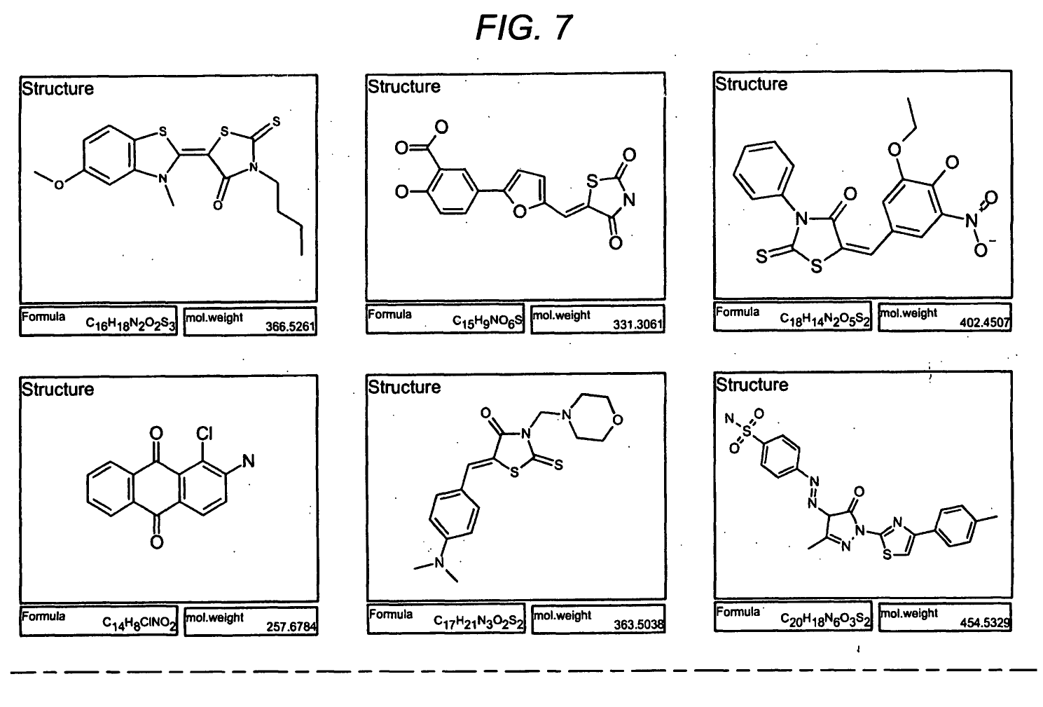

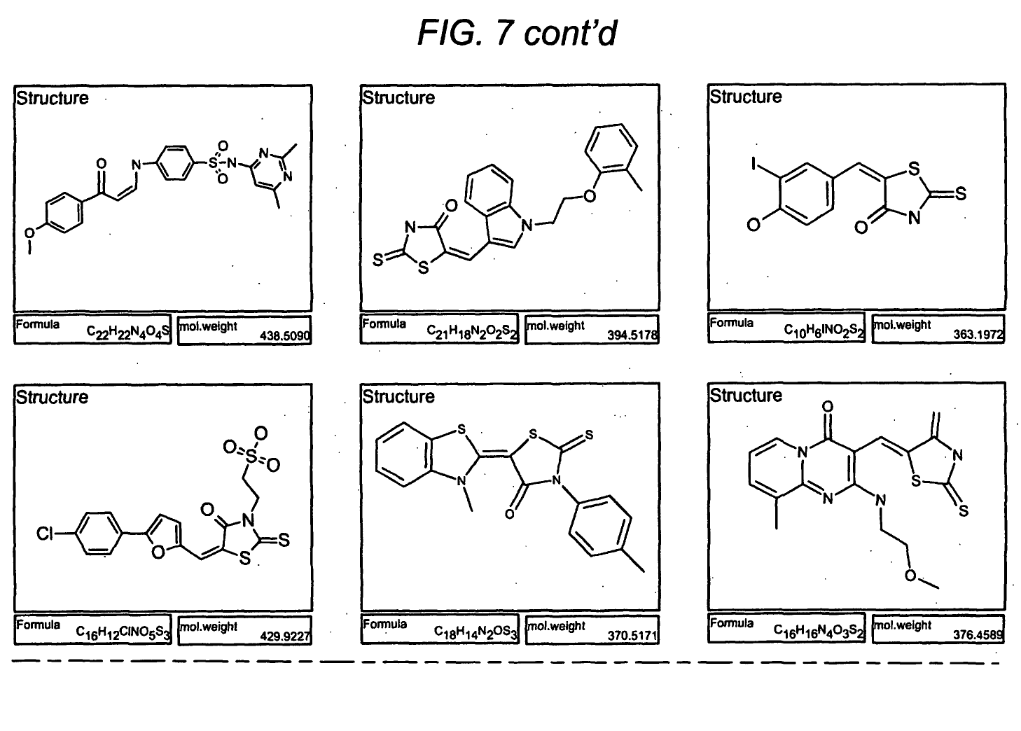

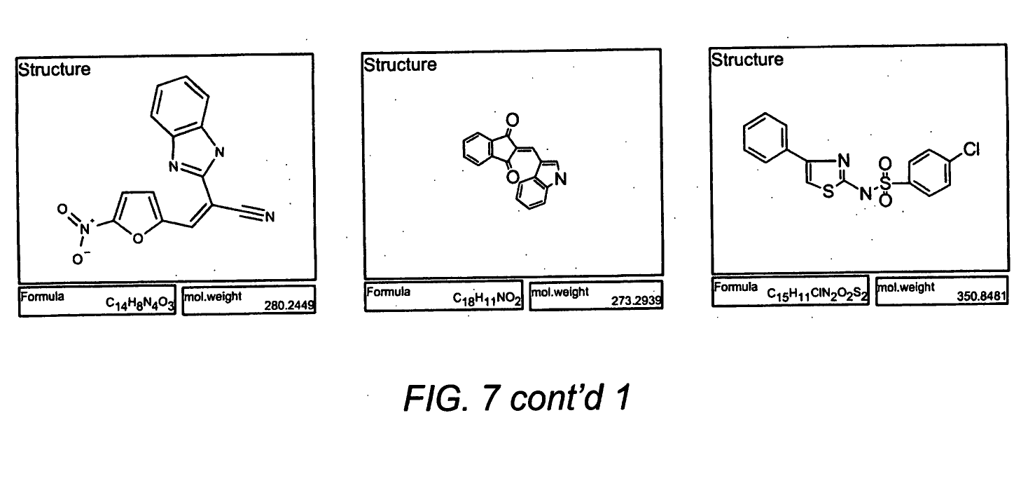

The structures of the compounds referred to throughout this document are shown in FIG. 7.

According to a first aspect of the present invention there is provided a compound for modulating a guanine nucleotide exchange factor for use in the preparation of an agent for the treatment of a neurological or neurodegenerative disease.

Preferably the guanine nucleotide exchange factor is for a protein belonging to the Rap family of small GTPases.

Most preferably, the guanine nucleotide exchange factor is for a protein belonging to the Rap 1 family of small GTPases.

Preferably, the compound is a cAMP effector.

Most preferably, the guanine nucleotide exchange factor is selected from the list EPAC 1 or EPAC 2.

Preferably, the compound is selected from the list:

C₁₆H₁₈N₂O₂S₃

C₁₅H₉NO₆S

C₁₈H₁₄N₂O₅S₂

C₁₄H₈CINO₂

C₁₇H₂₁N₃O₂S₂

C₂₀H₁₈N₆O₃S₂

C₂₂H₂₂N₄O₄S

C₂₁H₁₈N₂O₂S₂

C₁₀H₆INO₂S₂

C₁₆H₁₂CINO₅S₃

C₁₈H₁₄N₂OS₃

C₁₆H₁₆N₄O₃S₂

C₁₄H₈N₄O₃

C₁₈H₁₁NO₂

C₁₅H₁₁CIN₂O₂S₂

The structure of the compounds are shown in FIG. 7.

Optionally the compound is C₁₆H₁₈N₂O₂S₃

Optionally the compound is C₁₈H₁₄N₂O₅S₂

Optionally the compound is C₂₀H₁₆N₆O₃S₂

Optionally the compound is C₂₂H₂₂N₄O₄S

Optionally the compound is C₂₁H₁₈N₂O₂S₂

Optionally the compound is C₁₄H₈N₄O₃

Preferably, the compound is a functional analogue of any of the above-specified compounds.

Preferably an analogue of any of the above-specified compounds is any compound which shows >99% structural homology to the above-specified compounds.

Alternatively, an analogue of any of the above-specified compounds is any compound which shows >90% structural homology to the above-specified compounds.

Alternatively, an analogue of any of the above-specified compounds is any compound which shows >80% structural homology to the above-specified compounds.

Preferably the neurological or neurodegenerative disease is Alzheimer's Disease.

Alternatively the neurological or neurodegenerative disorder is Schizophrenia.

According to a second aspect of the present invention there is provided a compound for use as an EPAC selective inhibitor selected from the list:

C₁₆H₁₈N₂O₂S₃

C₁₅H₉NO₆S

C₁₈H₁₄N₂O₅S₂

C₁₄H₈CINO₂

C₁₇H₂₁N₃O₂S₂

C₂₀H₁₈N₆O₃S₂

C₂₂H₂₂N₄O₄S

C₂₁H₁₈N₂O₂S₂

C₁₀H₆INO₂S₂

C₁₆H₁₂CINO₅S₃

C₁₈H₁₄N₂OS₃

C₁₆H₁₆N₄O₃S₂

C₁₄H₈N₄O₃

C₁₈H₁₁NO₂

C₁₅H₁₁CIN₂O₂S₂

According to a third aspect of the present invention, there is provided a pharmaceutical composition comprising compounds that are able to modulate a guanine nucleotide exchange factor for the treatment of a neurological or neurodegenerative disease.

Preferably the guanine nucleotide exchange factor is for a protein belonging to the Rap family of small GTPases.

Most preferably, the guanine nucleotide exchange factor is for a protein belonging to the Rap 1 family of small GTPases.

Most preferably, the guanine nucleotide exchange factor is selected from the list EPAC 1 or EPAC 2.

Preferably, the compound is a CAMP effector.

Preferably, the compound is selected from the list:

C₁₆H₁₈N₂O₂S₃

C₁₅H₉NO₆S

C₁₈H₁₄N₂O₅S₂

C₁₄H₈CINO₂

C₁₇H₂₁N₃O₂S₂

C₂₀H₁₈N₆O₃S₂

C₂₂H₂₂N₄O₄S

C₂₁H₁₈N₂O₂S₂

C₁₀H₆INO₂S₂

C₁₆H₁₂CINO₅S₃

C₁₈H₁₄N₂OS₃

C₁₆H₁₆N₄O₃S₂

C₁₄H₈N₄O₃

C₁₈H₁₁NO₂

C₁₅H₁₁CIN₂O₂S₂

Optionally the compound is C₁₆H₁₈N₂O₂S₃

Optionally the compound is C₁₈H₁₄N₂O₅S₂

Optionally the compound is C₂₀H₁₈N₆O₃S₂

Optionally the compound is C₂₂H₂₂N₄O₄S

Optionally the compound is C₂₁H₁₈N₂O₂S₂

Optionally the compound is C₁₄H₈N₄O₃

Preferably, the compound is a functional analogue of any of the above-specified compounds.

Preferably an analogue of any of the above-specified compounds is any compound which shows >99% structural homology to the above-specified compounds.

Alternatively, an analogue of any of the above-specified compounds is any compound which shows >90% structural homology to the above-specified compounds.

Alternatively, an analogue of any of the above-specified compounds is any compound which shows >80% structural homology to the above-specified compounds.

Preferably the neurological or neurodegenerative disease is Alzheimer's Disease.

Alternatively the neurological or neurodegenerative disorder is Parkinson's disease, Huntington's disease or ALS.

Alternatively the neurological or neurodegenerative disorder is Schizophrenia.

According to a fourth aspect of the present invention, there is provided a method for identifying a compound for modulation of a guanine nucleotide exchange factor comprising the steps:
contacting a compound with the guanine nucleotide exchange factor
determining whether the compound activates or inhibits the guanine nucleotide exchange factor

Preferably, the method is suitable for identifying compounds suitable for use in the treatment of neurological or neurodegenerative disorders.

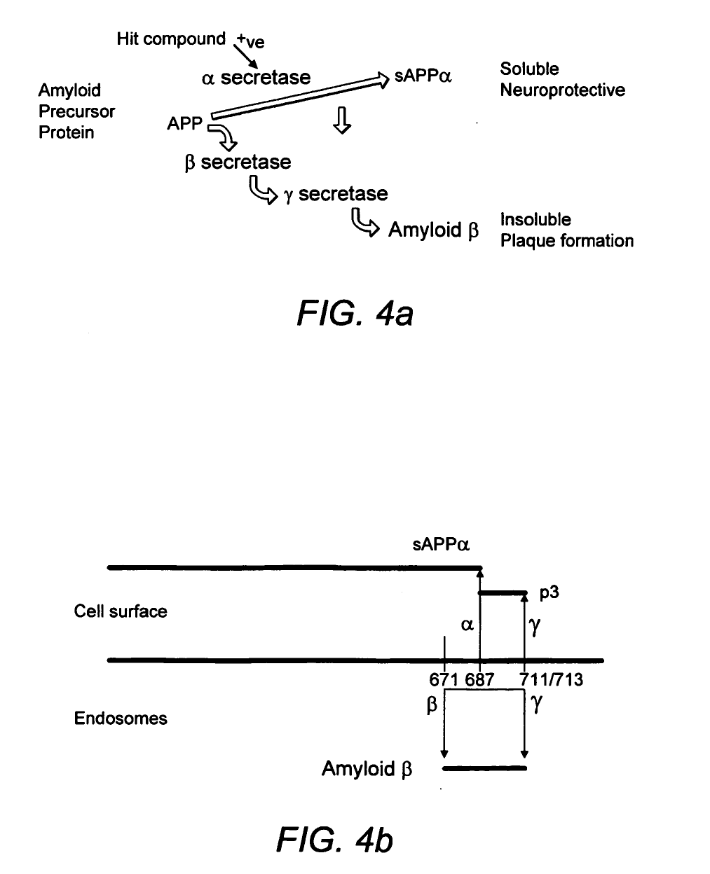

Optionally, the method for identifying compounds suitable for use in the treatment of neurological or neurodegenerative disorders also comprises the step:
identifying compounds which modulate sAPPα (soluble amyloid precursor protein a) secretion (see FIGS. 4a and 4b)

Optionally, the method for identifying compounds suitable for use in the treatment of neurological or neurodegenerative disorders also comprises the step:
identifying compounds which regulate phosphorylation of Tau protein in cells.

Preferably the neurological or neurodegenerative disorder is Alzheimer's.

Alternatively, the neurological or neurodegenerative disorder is Schizophrenia.

Preferably the guanine nucleotide exchange factor is for a protein belonging to the Rap family of small GTPases.

More preferably the guanine nucleotide exchange factor is for a protein belonging to the Rap 1 family of small GTPases.

Most preferably the guanine nucleotide exchange factor is selected from the list EPAC 1 or EPAC 2.

Preferably, the compound is a cAMP effector.

According to a fifth aspect of the present invention, there is provided a method of preparing a pharmacological composition for treating conditions linked to the up or down regulation of a guanine nucleotide exchange factor, which comprises:
a) identifying a compound which can modulate the guanine nucleotide exchange factor by contacting said compound with the guanine nucleotide exchange factor, and
b) formulating the compound identified in step a) as a modulator of the guanine nucleotide exchange factor into a pharmaceutical composition by mixing with a pharmaceutically acceptable carrier or diluent.

Optionally, the method of preparing a pharmacological composition also comprises the step (carried out prior to step b):
identifying compounds which modulate sAPPα secretion

Optionally, the method of preparing a pharmacological composition also comprises the step (carried out prior to step b):
identifying compounds which regulate phosphorylation of Tau protein in cells.

Throughout this document a guanine nucleotide exchange factor for Rap is any protein that elevates the exchange of GDP for GTP from Rap by direct physical interaction between the guanine nucleotide exchange factor and Rap.

Throughout this document EPAC 1 can be defined by the sequence held under Accession number AF103905.

Throughout this document EPAC 2 can be defined by the sequence held under Accession number NM_{—}007023.

Throughout this document, the term “Alzheimer's” should be taken to cover all dementias of the Alzheimer's type, including pre-senile dementia and also all dementias that do not have an obvious organic cause, such as stroke, brain damage or alcohol. The term should also be considered to cover any disease states which show the pathological changes of amyloid plaques, consisting of amorphous extra-cellular deposits of beta amyloid protein or neurofibrillary tangles which comprise filaments of a phosphorylated form of protein normally associated with intra-neuronal microtubules.

In researching disease targets for therapeutic intervention of brain disorders such as Alzheimer's Disease, the inventors carried out a study using microarray technology to look at the activity of genes involved in cyclic nucleotide signalling. As a result, it was discovered by the inventors that EPAC 1 and EPAC 2 genes (sequence listings for which are included as Sequence ID 1 and Sequence ID 2) are strongly up-regulated and down-regulated respectively in Alzheimer's Disease.

The cyclic nucleotide signalling cascade is a powerful controller of many cellular functions. Chemicals capable of modifying this system have been shown to have beneficial effect upon many disease states. However, EPACs have only recently been discovered, and their discovery identified a new way for cyclic AMP to exert effect on the cell. FIG. 1 summarises the action of EPACs on cyclic AMP. These cyclic AMP regulated guanine nucleotide exchange factors are widely expressed, and therefore have possible implications in a wide variety of cellular functions

Experimental Evidence Showing the Link Between EPAC 1 and EPAC 2 and Alzheimer's Disease

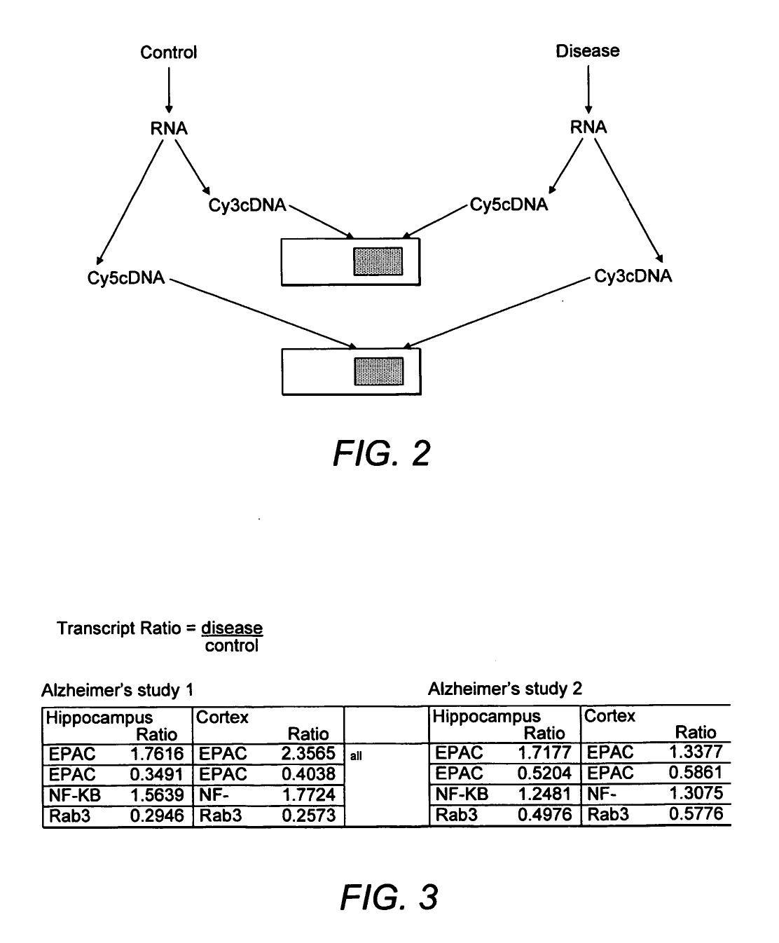

The following procedure was carried out in order to show the link between EPAC1 and EPAC2 and Alzheimer's (a basic overview of the gene array is shown in FIG. 2):

Set up the following cDNA synthesis for gene chip using total cell RNA (10-25 μg). 2 tubes per gene chip slide (i.e. 1 for Cy3 and 1 for Cy5) are required, 2 chips per experiment where the Cy dyes used for the control and diseased RNA are different for each gene chip and are swapped between the 2 gene chips. Tube 1 and 2 use RNA from control sample, tubes 3 and 4 use RNA from disease samples

| | | |
|---|---|---|
| Total RNA (1 μg/μl) | 23 | μl |
| Random Hexamers (3 μg/μl) | 4 | μl |
| Sub-Total | 27 | μl |

Incubate at 70° C. for 10 min and place on ice for 2 min before adding the following to each tube

| | | |
|---|---|---|
| 5× buffer | 10 | μl |
| 0.1M DTT | 5 | μl |
| dNTPmix(25 mM dATP, GTP, TTP, 10 mM dCTP) | 1 | μl |
| 1 mM Cy3-dCTP (tubes 1 and 3) or Cy5-dCTP (tubes 2 and 4) | 5 | μl |
| Superscript II (200 U/μl) | 2 | μl |
| Final Total | 50 | μl |

Take a note of which tube has Cy3 and which has Cy5.

Incubate at 25° C. for 10 min then 42° C. for 5 hr minimum.

Add 10 μl of 1M NaOH then mix and incubate at 65° C. for 15 min.

Add 10 μl of 1M HCl.

Add 350 μl of buffer PB (Qiagen supplied) and transfer to QIAquick™ column.

Centrifuge 13,000 rpm, for 1 min. at room temperature (r.t).

Add 0.75 μl of Buffer PE to QIAquick™ column

Centrifuge 13,000 rpm, for 1 min. at r.t.

Discard the flow-through

Centrifuge 13,000 rpm, for 1 min. at r.t.

Place QIAquick™ column into a clean 1.5 ml microfuge tube

Add 30 μl elution buffer to the centre of the QIAquick™ column

Let stand for 1 min.

Centrifuge 13,000 rpm, for 1 min. at room temperature.

Add 30 μl elution buffer to the centre of the QIAquick™ column

Let stand for 1 min.

Centrifuge 13,000 rpm, for 1 min. at r.t.

Take DNA solution (total 60 μl) and dry sample in a speed vac. (30° C./50-60 min.) until almost completely dry

Prewarm gene frame hybridization buffer to 65° C.

Add 110 μl of prewarmed hybridization buffer to dried cDNA pellets and mix well with pipette avoiding air bubbles

Combine Cy5 with Cy3 for control and test (tubes 1+4 and tubes 2+3) and mix well

Re-heat mixture to 65° C. for 2 min and pipette up and down again several times

Spin down in centrifuge (13,000 rpm/30 sec).

Apply gene frame from MWG to glass slides (as per manufacturers instructions). First separate an individual gene frame by cutting along the perforations.

Carefully remove the thick polyester sheet exposing the frame along 1 side.

Stick the frame around the spotted array on the glass slide.

Heat the hybridization mixture in a heating block at 95° C. for 3 min.

On ice for 30-60 sec.

Remove the thin polyester backing sheet from gene frame Apply the hybridization mixture to microarray along the edge of 1 side of the gene frame

Carefully apply polyester coverslip over gene frame from side where the mixture was applied to the opposite side avoiding trapping air bubbles in the process.

Incubate at 42° C. for 16-24 hours. Use shaking incubator (140 rpm).

Before washing the gene chip, preheat all buffers to 30° C. Wash microarray in washing buffer 1(2×SSC, 0.1% SDS) for 5 min. at r.t. with gentle agitation.

Wash microarray in washing buffer 2(0.5×SSC) for 5 min. at r.t. with gentle agitation. (Twice)

Drain off the excess water by gently dabbing the edge of the slide with a paper towel and leave to dry in air in dark. If array scan is particulate, re-wash and dry the array by placing in a 50 ml centrifuge and centrifugation twice in a bench to centrifuge at 1,400 rpm for 5 min.

The gene chip results indicated that EPAC 1 was up regulated in Alzheimer's disease by an average maximum factor of 2.4 fold and EPAC 2 was down regulated by and average maximum factor of 2.9 fold. Similar results were obtained when the inventors conducted a second study using completely different pools of Alzheimer's patients and controls. Both studies used the hippocampus and frontal cortex of the brain, the regions which are know to show the greatest degree of pathology in Alzheimer's. As a further control the cerebellum from the brains of study 2 were tested, as this region shows some resistance to damage from Alzheimer's disease. In contrast to the results obtained from the hippocampus and frontal cortex regions, the cerebellum of Alzheimer's patients showed a slight decrease (1.3 fold) in EPAC 1 and no detectable difference in EPAC 2. This showed that the large up regulation of EPAC 1 and down regulation of EPAC 2 in the frontal cortex and hippocampus of Alzheimer's patients is related to the pathology of the disease. Although the magnitude of the gene expression change detectable using gene array technology is only semi quantitative, the changes seen in the EPACs are comparable with those of control genes on the chip already known to change in Alzheimer's disease.

FIG. 3 is a table that indicates the changes that were noted by the inventors in Alzheimer's disease. It shows up-regulation of EPAC 1 and down-regulation of EPAC 2. It also shows changes to other genes that are already known to be involved in Alzheimer's, which further supports the validity of the results.

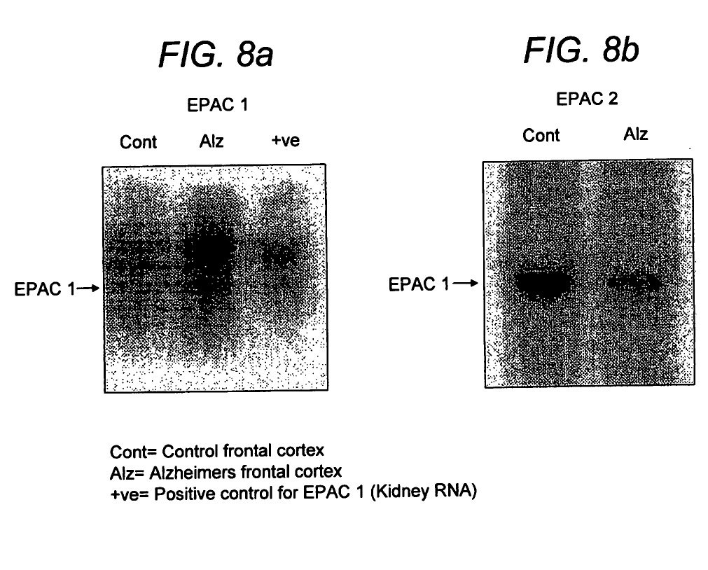

Northern blot analysis has also been carried out to confirm the results. The results of which can be seen in FIG. 8a (EPAC 1) and 8b (EPAC 2).

Using Guanine Nucleotide Exchange Factors, Such as EPAC 1 and EPAC 2 to Screen for Compounds for Use in the Treatment of Neurological Disorders

In the preferred embodiment, EPAC 1 and EPAC 2 are used to screen for compounds that may be useful in the treatment of Alzheimer's Disease and other neurological and neurodegenerative disorders. Below we have detailed potential screening methods that can be applied to identify compounds that either activate and/or inhibit EPACs.

Measuring cAMP Competition

This assay depends on compounds competing with cAMP for binding to the specific cAMP binding sites found on EPACs. In the presence of radio-labelled cAMP, EPAC proteins will become radio-labelled. Compounds, which compete with cAMP for binding to EPAC can therefore be detected as they will reduce the amount of radio-labelled protein. The amount of radio-labelled protein can be detected by either measuring the supernatant fraction after precipitating out free radio-labelled cAMP with charcoal, or by immobilising EPAC to protein binding filters and washing off any unbound cAMP.

Preferred Screen

The particular library screen that is preferred by the inventors was carried out using EPAC 1. The screen can be used on any appropriate library and could be modified for use with EPAC 2 or any other guanine nucleotide exchange factor for a protein belonging to the Rap family of small GTPases. The screen assesses the ability of test compounds to inhibit the binding of [3H]-cyclic AMP to EPAC 1 (Exchange Protein directly Activated by Cyclic AMP). EPAC 1 gene up-regulated in Alzheimer's therefore inhibition of activation by binding of cyclic AMP may have potential therapeutic effects in this disease area.

The steps are as follows:
1. Thaw library plates if required and remove 10 μl to a fresh daughter plate.
2. add 90 μl 15% DMSO to daughter plate
3. transfer 10 μl from daughter plates to assay plates
4. Add 10 μl 15% DMSO to row 1 A-F
5. Add 0.5M DTE to buffer E (50 mM Tris (6.07 g/l), 50 m MNaCl (2.92 g/l), 5% v/v glycerol (50 ml/l)pH 7.6) to make a final concentration of 5 mM.
6. Dilute 10 Mm cold cyclic AMP to 1 mM (add 900 ul 15% DMSO to 100 ul of 10 mM stock). Add 10 μl cyclic AMP to wells 1 G and H
7. Make up [3H] cyclic AMP (1.5 μCu/ml of 2704 Cu/mMol) and add 50 μl to all wells in assay plate
8. Re-suspend EPAC solution then dilute in buffer E. Add solution to small beaker and stir gently with stirrer bar. Add 50 μl/well to rows 1 to 11
9. Incubate on plate shaker at room temperature for 20 mins.
10. Place new filter plate in a cell harvester (i.e a Packard™ cell harvester)
11. Pre wash with 300 μl/well buffer. Add 30 ml in buffer tray and aspirate.
12. Place assay plate on bottom tray and aspirate.
13. Wash×3.
14. Open harvester and bottom seal filter plate
15. Take filter plate out of harvester and add 50 μl per well Microscint 20™
16. Shake plate on plate shaker for 15 mins
17. Read on Topcount™

The standard procedure uses a fusion protein of GST and EPAC (1 or 2) cAMP binding domain immobilised on glutathione beads.

The following compounds were found using EPAC screens:

| | EPAC 1 (n = 2) | EPAC 2 (n = 2) | (IC₅₀ μM) |
|---|---|---|---|
| C₁₆H₁₈N₂O₂S₃ | 4.3 | 19.3 | |
| C₁₅H₉NO₆S | 1.5 | 0.9 | |
| C₁₈H₁₄N₂O₅S₂ | 16.2 | 9.35 | |
| C₁₄H₈CINO₂ | 41 | 66 | |
| C₁₇H₂₁N₃O₂S₂ | 16 | 24.8 | |
| C₂₀H₁₈N₆O₃S₂ | 12.3 | 12.1 | |
| C₂₂H₂₂N₄O₄S | 162 | 284 | |
| C₂₁H₁₈N₂O₂S₂ | 16 | 14 | |
| C₁₀H₆INO₂S₂ | 23 | 30.5 | |
| C₁₆H₁₂CINO₅S₃ | 4 | 3 | |
| C₁₈H₁₄N₂OS₃ | 15 | 11.9 | |
| C₁₆H₁₆N₄O₃S₂ | 25 | 28.5 | |
| C₁₄H₈N₄O₃ | 20 | 26.9 | |
| C₁₈H₁₁NO₂ | 14 | 5.25 | |
| C₁₅H₁₁CIN₂O₂S₂ | 53 | 26.5 | |

Detecting Activated Rap with Radio-Labelled GTP

This assay relies on the fact that Rap protein exchanges GDP for GTP when activated by active EPAC. By using radio-labelled GTP, the activity of EPAC can be measured by detecting the amount of radio-labelled Rap. The amount of radio-labelled protein can be detected by measuring the supernatant fraction after precipitating out free radio-labeled GTP with charcoal, or by immobilising EPAC to protein binding filers and washing off any unbound GTP.

Fluorescence Transfer

This assay relies on the fact that Rap binds to Ral. This interaction is used to facilitate the transfer of electrons between fluorescent tags fused to these proteins in such a way that exciting the tag on Rap by chemical biolumnesence (BRET) or laser fluoresence (FRET) methods, will cause the tag on Ral to fluoresce or luminance. Fluorescence only takes place when Rap and Ral are tightly bound after EPAC activation of Rap, and therefore detection of the fluorescence can be used to determine EPAC activity.

GTP Analog tnpGTP

tnpGTP changes is fluorescence when bound to a protein using the same principle as described in the detection of activated Rap with radio-labelled GTP. A simple measurement in the change of fluorescence would allow the amount of activated Rap to be measured, which itself would give an indication of EPAC activity.

Ral GDS/RAP ELZSA Method

This methodology relies on the binding of EPAC activated Rap to Ral GDS. The bound Rap can then be quantified using anti RAP antibodies. The detection system can be based on western blotting, ELISA or any other appropriate method, such as Beadalite™/Luminex™.

Surface Plasmon Resonance

This method relies on binding EPAC to a surface of a chip for use on a machine which can measure the surface plasmon resonance (SPR) on the chip (for example a BIAcore™ machine). Compounds which bind to EPAC can be detected as changes in the SPR after they have passed over the chip.

Whole Cell

When EPAC activates the Ryanodine receptor in intact cells, it causes an influx of Ca²⁺. This activation can be detected using bioprobes, such as Fura 2 or Fluo 3. Raising intracellular cAMP by stimulating the cells with forskolin or caffeine will cause activation of EPAC. EPAC inhibitors can then be detected by measuring how much they inhibit the release of Ca²⁺. This assay can be expanded to included any protein activated by EPAC and any bioprobe able to detect the activation.

Alpha Screen

This proximity assay from P&E Bioscience relies on fluorescence transfer between beads coupled to proteins which interact given a particular event. EPAC activates Rap and thereby allows its interaction with Ral GDS. Therefore, labelling Rap and Ral GDS with alpha screen beads would provide the basis for the detection of EPAC activity.

Screening of Lead Compounds to Determine their Usefulness in the Treatment of Alzheimer's

Amyloid Cell Based Assay

As mentioned previously, amyloid plaques are found in the brains of Alzheimer's sufferers. These amyloid plaques have been found to occur when amyloid precursor protein (APP) is converted to insoluble amyloid β rather than the soluble neuro-protective sAPPα found in normal brain pathology. FIG. 4a shows the alternative processing pathways of the amyloid precursor protein, with FIG. 4b showing the alternative splice variants of APP.

This means that lead compounds that are identified as being modulators of EPAC can be further screened using an amyloid cell based assay with the following steps:
use IMR 32 Neuroblastoma cell line
serum starve cells using standard techniques and protocols
add test compound
measure level of sAPPα secretion

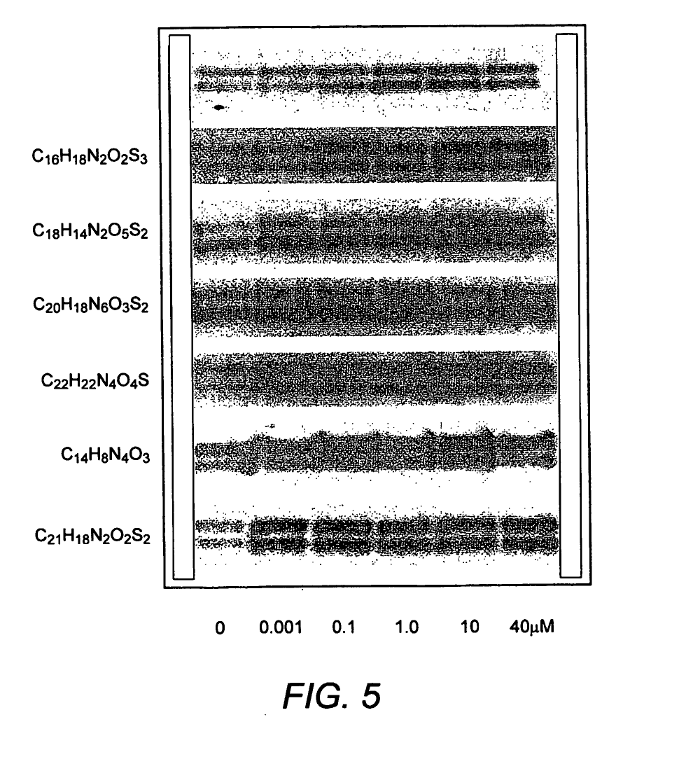

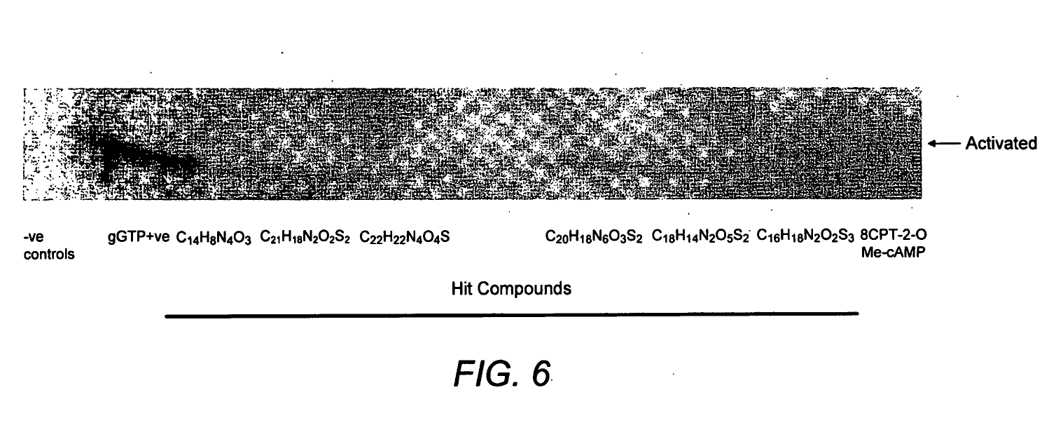

Results of an APPα induction screen are also shown in FIG. 5.

Phospho-Tau Assay

A similar screen can be used to look at the issue of neurofibrillar tangles. As with amyloid plaques, neurofibrillar tangles are associated with Alzheimer's. Neurofibrillar tangles occur when Tau protein is hyperphosphorylated. The assay could be a secondary screen or could be based on a phospho-Tau animal model.

Any lead compound that is identified can be screened against a panel of enzymes to test its specificity for EPAC. Enzymes that will be included in this panel will be PKA, cAMP gated ion channels and members of all human PDE families.

It can be seen that the present invention provides a number of benefits. In particular, it provides an important therapeutic target and chemical leads for the treatment of Alzheimer's and other neurological and neurodegenerative disorders. It also provides a method of screening for possible therapeutic compounds. This is the first indication of a link between EPAC 1 and EPAC 2 and neurodegenerative disorders such as Alzheimer's.

It will be appreciate by a person skilled in the art that the above embodiments have been described by way of example only, and should not be considered limiting. There are various alterations and modifications that are possible without departure on the scope of the invention as defined by the appended claims. In particular, alternative screening methods may be used other than those described in the example embodiments.

We acknowledge the Canadian Brain Tissue Bank, University of Toronto, UHN-Toronto Western Hospital, 399 Bathurst St., Fell Wing 5-222 A & B, Toronto, ON MST 2S8 for the provision of all our Alzheimer's brain specimens.

1. A compound for modulating a guanine nucleotide exchange factor for use in the preparation of a agent for the treatment of a neurological or neurodegenerative disease.

2. A compound as in claim 1, wherein the guanine nucleotide exchange factor is for a protein belonging to the Rap family of small GTPases.

3. A compound as in claim 1, wherein the guanine nucleotide exchange factor is for a protein belonging to the Rap 1 family of small GTPases.

4. A compound as in claim 1, wherein the compound is a cAMP effector.

5. A compound as in claim 1, wherein the guanine nucleotide exchange factor is selected from the list EPAC 1 or EPAC 2.

6. A compound as in claim 1, which is selected from the list:
C₁₆H₁₈N₂O₂S₃
C₁₅H₉NO₆S
C₁₈H₁₄N₂O₅S₂
C₁₄H₈CINO₂
C₁₇H₂ₗN₃O₂S₂
C₂₀H₁₈N₆O₃S₂
C₂₂H₂₂N₄O₄S
C₂₁H₁₈N₂O₂S₂
C₁₀H₆INO₂S₂
C₁₆H₁₂CINO₅S₃
C₁₈H₁₄N₂OS₃
C₁₆H₁₆N₄O₃S₂
C₁₄H₈N₄O₃
C₁₈H₁₁NO₂
C₁₅H₁₁CIN₂O₂S₂.

7. A compound as in claim 1, which is C₁₆H₁₈N₂O₂S₃.

8. A compound as in claim 1, which is C₁₈H₁₄N₂O₅S₂.

9. A compound as in claim 1, which is C₂₀H₁₈N₆O₃S₂.

10. A compound as in claim 1, which is C₂₂H₂₂N₄O₄S.

11. A compound as in claim 1, which is C₂₁H₁₈N₂O₂S₂.

12. A compound as in claim 1, which is C₁₄H₈N₄O₃.

13. A compound, which is a functional analogue of a compound as claimed in claim 6.

14-16. (canceled)

17. A compound as in claim 1, where the neurological or neurodegenerative disease is selected from the group consisting of Alzheimer's Disease, Parkinson's disease, Huntington's disease, ALS and Schizophrenia.

18. (canceled)

19. A compound for use as a EPAC selective inhibitor, wherein the compound is a compound as claimed in claim 6.

20. A pharmaceutical composition comprising a theraputically effective amount for treating a neurological or neurdegenerative disease in a subject in need thereof of a compound as claimed in claim 1 and a pharmaceutically acceptable carrier therefore.

21-37. (canceled)

38. A method for identifying a compound for modulation of a guanine nucleotide exchange factor as recited in claim 1, comprising the steps:
contacting a compound with the guanine nucleotide exchange factor; and
determining whether the compound activates or inhibits the guanine nucleotide exchange factor.

39-50. (canceled)

51. A method for treating a neurological or neurodegenerative disease in a subject in need thereof comprising administering to said subject a theraputically effective amount of a compound as claimed in claim 1 to thereby treat said subject.

52-56. (canceled)

57. A method as in claim 51, wherein the neurological or neurodegenerative disorder is selected from the group consisting of Alzheimer's, Schizophrenia Parkinson's disease, Huntington's disease and ALS.

58. (canceled)

59. A method as claimed in claim 51, wherein said compound is a compound as claimed in claim 6.


SEQUENCE LISTING (2 sequences)

SEQ ID NO: 1 - DNA, 2646 bp, Homo sapiens
atggtgttgagaaggatgcaccggccccgaagctgctcctaccagctgctgctggagcaccagcgtccgagctgcatccaggggctgcgctggacaccactcaccaacagcgaggagtccctggatttcagcgagagcctggagcaggcctccacagagcgggtgctcagggctgggaggcagctgcatcggcatctgctggccacctgcccaaacctcacccgagaccggaagtaccaccttaggctctatcggcagtgctgctctggccgggagctggtggatgggatcttggccctgggacttggggtccattcccggagccaagttgtgggaatctgccaggtgctgctggatgaaggtgccctctgccatgtgaaacacgactgggccttccaggaccgagatgcccaattctaccggttccccgggcccgagcccgagcccgtgggaactcatgagatggaggaggagttggccgaagctgtggccctgctctcccagcgggggcctgacgccctgctcactgtggcacttcgaaagcccccaggtcagcgcacggatgaagagctggacctcatctttgaggagctgctgcacatcaaggctgtggcccacctctccaactcggtgaagcgagaattagcggctgttctgctctttgaaccacacagcaaggcagggaccgtgttgttcagccagggggacaagggcacttcgtggtacattatctggaagggatctgtcaacgtggtgacccatggcaaggggctggtgaccaccctgcatgagggagatgattttggacagctggctctggtgaatgatgcaccccgggcagccaccatcatcctgcgagaagacaactgtcatttcctgcgtgtggacaagcaggacttcaaccgtatcatcaaggatgtggaggcaaagaccatgcggctggaagaacatggcaaagtggtgctggtgctggagagagcctctcagggcgccggcccttcccgacccccaaccccaggcaggaaccggtatacagtgatgtctggcaccccagagaagatcctagagcttctgttggaggccatgggaccagattccagtgctcatgacccaacagagacattcctcagcgacttcctcctgacccacagggtcttcatgcccagcgcccaactctgcgctgcccttctgcaccacttccatgtggagcctgcgggtggcagcgagcaggagcgcagcacctacgtctgcaacaagaggcagcagatcttgcggctggtcagccagtgggtggccctgtatggctccatgctccacactgaccctgtggccaccagcttcctccagaaactctcagacctggtgggcagggacacccgactcagcaacctgctgagggagcagtggccagagaggcggcgatgccacaggttggagaatggctgtgggaatgcatctcctcagatgaaggcccggaacttgcctgtttggctccccaaccaggacgagccccttcctggcagcagctgtgccatccaagttggggataaagtcccctatgacatctgccggccagaccactcagtgttgaccctgcagctgcctgtgacagcctccgtgagagaggtgatggcagcgttggcccaggaggatggctggaccaaggggcaggtgctggtgaaggtcaattctgcaggtgatgccattggcctgcagccagatgcccgtggtgtggccacatctctggggctcaatgagcgtctctttgttgtcaacccacaggaagcgcatgagctgatcccacaccctgaccagctggggcccactgtgggctctgctgaggggctggacctggtgagtgccaaggacctggcaggccagctgacggaccacgactggagcctcttcaacagtatccaccaggtggagctgatccactatgtgctgggcccccagcatctgcgggatgtcaccaccgccaacctggagcgcttcatgcgccgcttcaatgagctgcagtactgggtggccaccgagctgtgtctctgccccgtgcccggcccccgggcccagctgctcaggaagttcattaagctggcggcccacctcaaggagcagaagaatctcaattccttctttgccgtcatgtttggcctcagcaactcggccatcagccgcctagcccacacctgggagcggctgccccacaaagtccggaagctgtactccgccctcgagaggctgctggatccctcatggaaccaccgggtataccgactggccctcgccaagctctcccctcctgtcatccccttcatgccccttcttctcaaagacatgaccttcattcatgagggaaaccacacactagtggagaatctcatcaactttgagaagatgagaatgatggccagagccgcgcggatgctgcaccactgccgaagccacaaccctgtgcctctctcaccactcagaagccgagtttcccacctccacgaggacagccaggtggcgaggatttccacatgctcggagcagtccctgagcacccggagtccagccagcacctgggcttatgtccagcagctgaaggtcattgacaaccagcgggaactctcccgcctctcccgagagctggagccatga

SEQ ID NO: 2 - DNA, 4278 bp, Homo sapiens
cgggaggagcggggtccgcgcggcggacgaggcgggggcaggaggcgcaggcagagcgagcgcgggaggtcgccgcagcacagaggacaccgcgcgccgccgctcaacatggtcgctgcgcacgctgcccattcttcctcctctgccgagtggatcgcctgcctggataaaagaccactggagcgatccagcgaagatgtggatataatcttcactcgactgaaagaagttaaagcttttgagaaatttcacccaaatctccttcatcagatttgcttatgtggttattatgagaatctggaaaagggaataacattatttcgccagggtgatattggaacaaactggtatgctgtcctggcagggtctttggatgttaaagtatctgagaccagcagtcaccaggatgctgtgaccatctgtaccctgggaattgggacggcctttggagagtccattctggacaacacaccccgccatgcaaccatcgttaccagggagagcagtgaactgctccgcatcgagcagaaggacttcaaggcactatgggagaaatatcgacagtatatggcaggacttctggctcctccttatggtgttatggaaacgggctctaacaatgacaggattcctgacaaggagaacacacctctcattgaacctcacgttcctcttcgtcctgctaacaccattaccaaggtcccttcagagaagatcctcagagctggaaaaattttacgaaatgccattctctctcgagcacctcacatgataagagatagaaaataccacctaaagacatacagacaatgctgtgtgggaactgaactggtggactggatgatgcagcagacaccatgtgttcactcccggactcaagctgttggcatgtggcaagtcctgttagaagatggtgttctcaaccacgtggaccaggagcaccatttccaagacaaatatttattctatcgatttctggatgatgagcacgaggatgcccctttgcctactgaggaggagaagaaggagtgtgatgaggagctccaggacaccatgctgctgctgtcacagatgggccccgacgcccacatgaggatgatccttcgcaaaccacctggccagaggactgtggatgacctagagattatctatgaggagcttcttcatattaaagccttatcccatctttctaccacagtgaaacgagagttagcaggtgttctcatttttgagtctcacgccaaaggagggactgtgttgtttaaccagggggaagaaggtacctcctggtacattattctaaaaggatcagtgaatgtagtcatttacggcaagggtgtggtctgcaccctgcatgaaggagatgacttcggcaagttagcactagtgaatgatgccccacgagctgcctctatcgtcttacgagaagataactgccatttcttaagagtagacaaggaggatttcaaccggatcctaagggacgtggaggcgaatacagtcagacttaaagaacatgaccaagatgtcttggtgctggagaaggtcccagcagggaacagagcttctaatcaaggaaactcacagcctcagcaaaagtatactgtgatgtcaggaacacctgaaaaaattttagagcattttctagaaacaatacgccttgaggcaactttaaatgaagcaacagattctgttttaaatgactttattatgatgcactgtgtttttatgccaaatacccagctttgcccggcactggtggcccactaccacgcacagccttcacaaggtacagaacaggagaaaatggattatgccctcaacaataagaggcgagtcatccgcctggttctacagtgggctgccatgtatggagacctcctgcaagaggatgacgtatctatggccttcctggaggagttttatgtatctgtatcagatgatgcccggatgattgctgccctcaaggagcaactgccagagttggagaagattgtcaagcaaatctcagaagatgcaaaggcaccacaaaagaagcacaaggttcttttgcaacagttcaatacgggcgatgagagagcccagaagcgccagcctatccgcggctctgatgaagttctgtttaaggtctattgcatggaccacacctacacaaccattcgggtgccagtggccacttcggtgaaggaagtcatcagtgcagttgccgacaagctgggctccggggagggcctgatcatagtcaagatgagttccggaggagaaaaggtggtgctcaaacctaatgatgtttcagtatttacgacgctcaccattaatggacgcctgtttgcttgcccgcgagagcaattcgattcactgactcccttaccagaacaggaaggcccaactgttggaacagtgggaacttttgaactgatgagctccaaagatttagcataccagatgacaatttatgattgggaactcttcaactgcgtgcatgagctggagctaatctatcacacatttggaaggcataattttaaaaagaccacagcaaacttggatttgttcctgaggagatttaatgaaattcagttttgggtcgtcactgagatctgcctttgttctcagctcagcaagcgtgttcagctattaaaaaaatttattaagatagcagcccactgtaaggagtataaaaatctgaattccttttttgccatcgtcatgggactaagtaacattgctgtgagccgcttggcactaacgtgggagaaactgccaagcaagttcaagaagttctatgcggagtttgaaagtttaatggacccttcaaggaaccacagggcctacaggctgacagtagctaagctggaacctcctctcatccccttcatgcctttgctcattaaagatatgacatttactcatgaggggaacaagacgttcattgacaatctagtaaactttgaaaaaatgcgcatgattgcaaatacggccagaacagtgagatactacaggagccaacccttcaatcctgatgcagctcaagctaataagaaccatcaggatgtccggagttatgtacggcaattaaatgtgattgacaaccagagaactttatcacagatgtcacacagattagagcctcgtcgaccatagacatttcaaatgcccaaagcaacagtttgtctccagtccacaatctttcaaaaatgccatttatgctactactgactgtattgccactagagaattctacaaaacaagcaaaaacacatcctgagacacctcagggctgcattcagcttaccagctacctagcaagagaaggaattatttgacatgcctaaagcttacacactctagcttaggaatccccagtttgtggctaccctgtttcatttccacttgtgccatcttctttgctgaagtgttgaataaggcccacgtgaagagtttggtagaaatagccttgtcaagagaactagcaagcccctgacatttttttctaagagtgtttatgggatgagatgtgctacaaatgggatagatttggataaaattttaactcaacatcttgatttggagcctggggtttcgggaaggtgttgaggaatctatagaagtccaaattataggttgtattcttccatcttcattgtcttacctctgaaggaatagaacctgaccacattattatgaaacattatggctgtttatgtaatttagggaggactggtctgtaatttctgaatgtatatagaataatatttatgtttacaatgtaacttttcaaaatttacaaatcaggattatatacataagaattccactaagaaatgccaagattcttaaatttgccagcgttaaagtagaaaataacatttcagaagagcacaatatgcataaaacatttttcaaaattgaaatattttcctgggcattaaaaacctttactattggctacaaatttattgtacctgatgaaaacatattttctggacttaaatgttattacaaatatcttaattttcagtaattgttttgcactttcaaagattgtaaatagttcattcaatcaatggtatagagttatttatttgctacataatagatactgtgccaaataattactttttatttattttatttagtacgtaattttgaagtacattttttcctgttttcacaattagactacatttaatgtgtaggaattgtatgtatgtatatcttctgtaaataacatctagtatcttcactaaatataattgtcgaccgggaaaaaaaaaa